(19) United States
(12) Patent Application Publication
(10) Pub. No.: US 2021/0128643 A1
(43) Pub. Date: May 6, 2021

(54) MICRO-ORGANISMAL PRODUCT AND METHOD FOR TREATING SICKLE CELL DISEASE

(71) Applicant: Seah H. Lim, Great Neck, NY (US)

(72) Inventor: Seah H. Lim, Great Neck, NY (US)

(21) Appl. No.: 17/071,956

(22) Filed: Oct. 15, 2020

Related U.S. Application Data
(60) Provisional application No. 62/928,080, filed on Oct. 30, 2019.
(60) Provisional application No. 62/935,933, filed on Nov. 15, 2019.

(51) Int. Cl. A61K 35/741; G01N 33/68; A61K 9/00; A61J 15/00; A61M 31/00

(52) U.S. Cl. CPC A61K 35/741; G01N 33/6893; A61K 2035/115; A61J 15/0003; A61M 31/00; A61K 9/0053

(57) ABSTRACT

The present disclosure provides a micro-organismal product and a method for treating a patient with sickle cell disease. In one aspect, the method comprises determining a concentration of one or more biological markers in serum of the patient indicative of breach of an intestinal barrier in sickle cell disease; and administering a micro-organismal product to the patient that restores intestinal permeability of the patient by, for example, upregulating tight-junctions of the patient's intestine.

### RELATED APPLICATIONS

This application claims the benefit of priority to U.S. Provisional Application No. 62/928,080, filed on Oct. 30, 2019, and U.S. Provisional Application No. 62/935,933, filed on Nov. 15, 2019, the entire contents of both of which are incorporated herein by reference for all purposes.

### TECHNICAL FIELD

The present disclosure relates to a micro-organismal product and a method for treating sickle cell disease (SCD). More particularly, the present disclosure relates to a micro-organismal product and a method for treating sickle cell disease by modifying intestinal permeability.

### BACKGROUND

Sickle cell disease (SCD) is a major public health problem. It affects millions of people worldwide. It is estimated that approximately 300,000 babies in the United States are born each year with SCD. It is a chronic illness and affected individuals experience recurrent Vaso-Occlusive Crisis (VOC), poor quality of life, and a shortened lifespan.

A study of the National Center for Health Statistics multiple-cause-of-death databases from 1979 to 2005 highlighted the magnitude of the adverse effects of SCD on life expectancy. In 2005 in the United States, the median life expectancy of SCD subjects was only 42 years for females and 38 years for males, compared to 77 years for females and 72 years for males in the general U.S. population. Although the mortality rate in SCD children decreased by 3% a year between 1979 and 2005, the adult mortality rate increased by 1% each year, higher than can be explained by an influx of younger patients into adulthood, and probably reflecting the limited accessibility to high quality medical care in adult subjects with SCD. As lifespan extends into adulthood, end-organ damage occurs, affecting the kidneys, brains, lungs, liver, and eyes.

Around 100,000 Americans are affected by SCD. They impose high healthcare utilization. According to the Healthcare Cost and Utilization Project (HCUP), a total of 83,149 hospitalizations were incurred by adult SCD in 2004 in the United States, with the total cost estimated at about $488 million. A benchmark study found a mean annual rate of 1.52 admissions per SCD patients and 1.08 treat-release Emergency Department visits per year. Healthcare utilization was highest among the 18-30-year-old group. SCD, therefore, poses a major public health concern.

Due to repeated hospitalizations, disabling symptoms, and progressive end-organ damage, individuals with SCD are subjected to an inexorable decline in health and quality of life, and financial instability that amplifies the health disparity in these ethnic groups of patients. Given the disease burden and how it impacts not only the affected individuals but also the society at large, any actionable findings that allow the design of approaches to improve the quality and quantity of life and change the disease outcome of SCD patients is highly significant.

### SUMMARY

The inventor of the present disclosure has found that patients with SCD have abnormal intestinal composition that is associated with the increased intestinal permeability in these patients. Because of the increased intestinal permeability, these patients exhibit higher levels of translocation of bacteria/bacterial products into the systemic circulation to activate and age the neutrophils. Both activated and aged neutrophils are responsible for the pathogenesis of the complications associated with SCD. Since Akkermansia muciniphila, its extracellular vesicles, and its products/components are capable of upregulating tight-junction (TJ) formation, they can be used to treat SCD. Other probiotic bacteria including but not limited to Lachnoclostridium, Alistipes, and Pseudobutyrivibrio, are believed to have similar effects. Modes of treatment include, but not limited to, the use of endoscopy, nasogastric tube, capsules, or tablets.

In one aspect, the present disclosure provides a method for treating a patient with sickle cell disease. The method comprises determining a concentration of one or more biological markers in serum of the patient indicative of breach of an intestinal barrier in sickle cell disease; and administering a micro-organismal product to the patient that restores intestinal permeability of the patient.

In another aspect, the present disclosure provides a method for treating a patient with sickle cell disease. The method comprises determining intestinal microbial composition of the patient with sickle cell disease; and administering a micro-organismal product to the patient, the micro-organismal product capable of restoring an intestinal microbial composition of the patient to a normal level.

In one embodiment, administering the micro-organismal product to the patient comprises administering probiotic bacteria to the patient.

In one embodiment, the probiotic bacteria comprise one or more of Akkermansia muciniphila, Lachnoclostridium, Alistipes, and Pseudobutyrivibrio.

In one embodiment, administering the micro-organismal product to the patient comprises delivering the micro-organismal product using at least one of endoscopy, a nasogastric tube, a capsule, a drink, and a tablet.

In one embodiment, determining the concentration of said one or more biological markers comprises determining a concentration of one or more of intestinal fatty acid binding protein (iFABP), lipopolysaccharide (LPS), and L-selectin (CD62L).

In one embodiment, determining the intestinal microbial composition comprises determining abundance of one or more of bacterial genera.

In one embodiment, the bacterial genera include Pseudobutyrivibrio, Faecalibacterium, Subdoligranulum, Prevotella_9, Alistipes, and Escherichia-Shigella.

In one embodiment, administering the micro-organismal product to the patient comprises administering the micro-organismal product to upregulate tight-junctions of the patient's intestine.

### BRIEF DESCRIPTION OF THE DRAWINGS

FIG. 1 shows levels of serum intestinal fatty acid binding protein (iFABP) in SCD patients being significantly greater than that in the control group.

FIG. 2 shows levels of serum lipopolysaccharide (LPS) in SCD patients being significantly greater than that in the control group.

FIG. 3 shows levels of serum soluble L-selectin (CD62L) in SCD patients being significantly greater than that in the control group.

FIG. 4 shows that elevated levels of serum soluble CD62L predict a greater percentage of circulating aged neutrophils.

FIG. 5 shows that levels of serum LPS correlate positively with levels of soluble CD62L.

FIG. 6 shows that levels of serum LPS correlate negatively with levels of iFABP.

FIG. 7 shows that levels of serum iFABP correlate negatively with levels of CD62L.

FIG. 8 shows the vicious cycle of SCD.

### DETAILED DESCRIPTION

Observations in the clinic show that the number of sickled erythrocytes on the peripheral blood smear does not always correlate with symptoms of VOC, suggesting that, in addition to sickled erythrocytes, certain co-factors are needed for the pathogenesis of VOC. Many clinical and laboratory studies have implicated neutrophils as one of the co-factors. SCD patients have higher white cell counts (WBCs) than those without the disease. Patients with a WBC>15×10⁹/L are more likely to develop stroke and acute chest syndrome and die prematurely. In patients treated with hydroxyurea for SCD, reduction of the frequency of VOC occurs not only in those who have increases in fetal hemoglobin (HbF), but also in those who have decreases in neutrophil counts, irrespective of whether there are associated increases in the HbF levels, highlighting the role of neutrophils in the pathogenesis of VOC.

In addition to quantitative difference, neutrophils in SCD are also qualitatively distinct. The neutrophils show higher levels of activation molecules, e.g., CD64 and CD11b/CD18, and the sera elevated soluble CD62L, a marker of neutrophil activation. In SCD mice, combination of oral vancomycin, metronidazole, ampicillin and streptomycin decreases circulating aged neutrophils (CANs) and protects the mice from TNF a-induced fatal VOC. CANs and activated neutrophils are pivotal for the pathogenesis of VOC. Sickled erythrocytes are more likely to adhere to activated neutrophils than to endothelium. Immobilized neutrophils are, therefore, the niduses for sickled erythrocytes to attach to and cause VOC. The ability of the P-selectin antibody, crizanlizumab, to reduce the frequency of VOC without any change in the laboratory parameters for hemolysis further supports the role of neutrophils in VOC. These results point to the role of neutrophils as a co-factor in the pathogenesis of VOC and provide the basis for investigations into non-erythrocyte-related factors.

Gut permeability is a complex system of an anatomical barrier of intestinal wall and a barrier closely linked to the intestinal microbiota and elements of the mucosal immune system. The intercellular space between enterocytes is sealed by tight-junctions (TJs) that regulate the flow of water ions and small molecules. TJs are composed of proteins, such as, claudins, occludin, and tricellulin. This barrier is altered by changes in intestinal microbial composition, mucus layer alterations, and epithelial damage.

Hypoxia is a causative factor for epithelial damage. When the intestinal barrier is breached, increased luminal content and bacteria are translocated into the systemic circulation. Increased gut permeability has been implicated in the pathogenesis of various diseases. A balanced intestinal microbial composition helps to modulate the metabolic processes that influence gut permeability, due to effects on the production of short chain fatty acids (SCFAs) important for enterocyte development or through bacterial factors that directly affect TJ formation.

A previous study using genetically engineered mice shows that CANs are regulated by intestinal microbiota via TLR 2/4 and Myd88, both are receptors for pathogen-associated molecular patterns (PAMPs). Since VOC causes local hypoxia/reperfusion injury, SCD patients may have breach in their intestinal barrier, allowing an increase in the translocation of luminal bacteria and bacterial products into the systemic circulation to activate neutrophils.

The intestinal microbial composition of patients with SCD can be evaluated to determine whether the normal intestinal microbial composition is perturbed. Although no significant differences are found between SCD and sickle trait controls in the microbiota diversity or composition at the phylum level, when the top 15 genera are modelled (accounted for about 84% of the taxonomic abundance across all samples) and log-ratio transformed and treated the abundance as responses, six bacterial genera, especially Alistipes and Pseudobutyrivibrio, are found to be associated with SCD (see below TABLE 1), where Pseudobutyrivibrio, Faecalibacterium, Subdoligranulum, Prevotella 9, and Alistipes are lower in abundance, while Escherichia-Shigella is higher in abundance.

TABLE 1

| Genus | Phylum | Changes | P value |
|---|---|---|---|
| Pseudobutyrivibrio | Firmicutes | Lower | 0.05 |
| Faecalibacterium | Firmicutes | Lower | 0.11 |
| Subdoligranulum | Firmicutes | Lower | 0.16 |
| Prevotella_9 | Bacteroidetes | Lower | 0.07 |
| Alistipes | Bacteroidetes | Lower | 0.04 |
| Escherichia-Shigella | Proteobacteria | Higher | 0.11 |

Next, it is determined whether these six genera may be predictors of a set of clinical variables (e.g., Hb, HbF, LDH, WBC, neutrophil count, erythrocyte sedimentation rate and CRP). It appears that LDH shows the most significant association (adjusted R2=0.81, p=0.03) which positively correlates with Subdoligranulum, Escherichia-Shigella, Prevotella_9, and Faecalibacterium, and negatively correlates with Pseudobutyrivibrio and Alistipes. Among SCD subjects, higher abundance of Lachnoclostridium is positively associated with higher baseline Hb (p=0.01) and HbF (p=0.01), and lower baseline CRP (p=0.05) and total WBC (p=0.05), all of these being parameters of less severe disease. Others have similarly found altered intestinal microbial composition in SCD with increased abundance of Veillonella that correlates with frequency of VOC. These results suggest that changes in the intestinal microbial composition in SCD may influence the phenotype of the disease.

Aged and activated neutrophils are pivotal for the pathogenesis of VOC. Since neutrophil ageing is mediated through Myd88 and TLR 2/4 whose ligands are PAMPs produced by microbes and since intestinal microbial composition is altered, it follows that there must have been increased translocation of intestinal bacteria/bacterial products across the intestinal barrier into the systemic circulation in SCD. Therefore, while not wishing to be bound by theory, it is believed that, if SCD patients do in fact have gut injury and breach of intestinal barrier, SCD can be treated using intact (live or inactivated) Akkermansia muciniphila, its vesicles, or its products to improve the intestinal barrier and reducing the translocation of the bacteria/bacterial products.

Akkermansia muciniphila is a gram-negative strictly anaerobic micro-organism that belongs to the Verrucomicrobia phylum. It regulates intestinal TJs. Reduced abundance of Akkermansia muciniphila in the intestine has been associated with obesity and metabolic syndrome. A recent study administering live and pasteurized bacteria to obese human volunteers supports its safety for human use. Besides improvement in the parameters of metabolic syndrome, the study has also found that the circulating white blood cells and lipopolysaccharides (LPS) are lower in those patients treated with the pasteurized bacteria. Others have found that bacteria-free extracellular vesicles derived from Akkermansia muciniphila are also able to upregulate TJs, suggesting that Akkermansia muciniphila and its products can be a therapeutic agent for medical conditions associated with intestinal barrier breakdown.

Hypoxia-reperfusion injury causes tissue damage. Intestinal damage would, therefore, be expected if sickle cell VOC affects the splanchnic vasculature. Intestinal fatty acid binding protein (iFABP) is expressed in enterocytes of the small intestine. Since iFABP is released into the systemic circulation when there is intestinal damage, measurements of the serum iFABP in SCD would provide the evidence for intestinal injury due to VOC affecting the splanchnic vasculature.

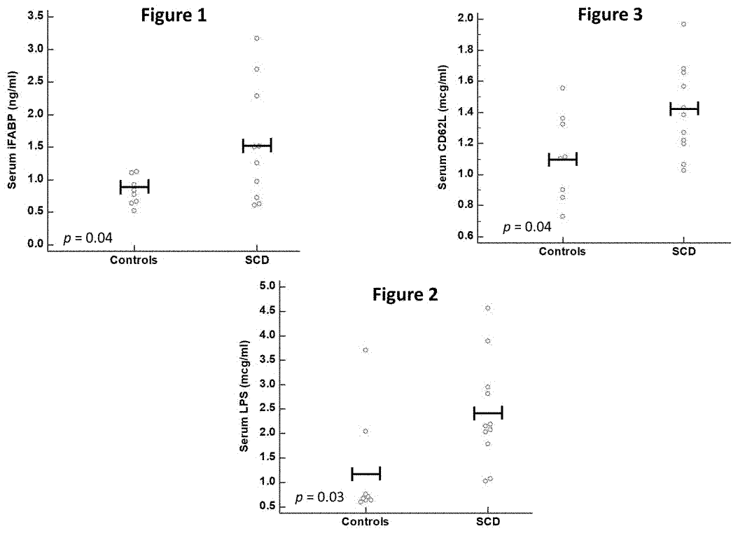

As shown in FIG. 1, serum iFABP in SCD individuals is found to be significantly higher than that in the control group (median: 1.38 ng/ml [range 0.61-3.17] vs 0.8 ng/ml [range 0.53-1.12]; p=0.04), providing the first evidence of intestinal injury in SCD. In addition, as shown in FIG. 2, it is found that translocation of bacterial products across the intestinal barrier into the systemic circulation is increased in SCD patients, as measured by the higher serum lipopolysaccharide (LPS) levels (median: 2.15 μg/ml [range 1.03-4.56] vs 1.2 μg/ml [range 0.60-3.70]; p=0.03). In keeping with the findings in a previous study, as shown in FIG. 3, SCD individuals in our study also has higher levels of soluble CD62L (median: 1.38 μg/ml [range 1.03-1.97] vs 1.11 μg/ml [0.73-1.55]; p=0.04), indicating the association between increased intestinal permeability, increased translocation of bacteria/bacterial products, and the presence of higher activated neutrophils in circulation.

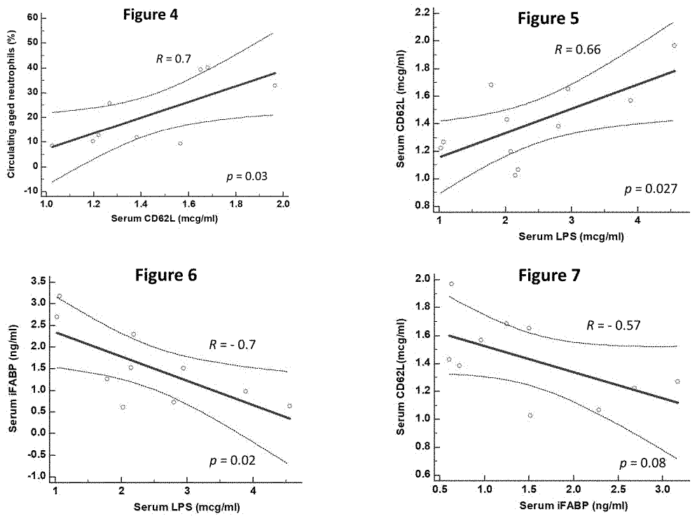

Since soluble CD62L in SCD is elevated, it is then determined how soluble CD62L correlates with the percentage of circulating aged neutrophils. As shown in FIG. 4, elevated serum soluble CD62L predicts a higher percentage of circulating aged neutrophils (R=0.7; p=0.03). This result supports the utilization of soluble CD62L as the surrogate for circulating aged neutrophils in our subsequent data analysis. As shown in FIG. 5, it is identified that serum LPS correlates positively with soluble CD62L (R=0.66; p=0.027), supporting the notion that LPS translocated across the intestinal barrier into the systemic circulating may be, at least in part, responsible for modulating circulating aged neutrophils in SCD.

Since elevated translocation of LPS across the intestinal barrier may be the result of increase in either gut permeability or intestinal microbial density, it is next determined the relationship between serum iFABP and LPS. If serum LPS is solely due to a compromised gut barrier, serum iFABP in SCD would be expected to correlate positively with LPS levels and percent of circulating aged neutrophils. As shown in FIG. 6 and to our surprise, however, a negative correlation between serum LPS and iFABP (R=−0.7; p=0.02) is observed. Similarly, as shown in FIG. 7, a negative correlation trending towards significance occurs between serum iFABP and soluble CD62L=−0.57; p=0.08). These negative correlations may be accounted for by one of two explanations, or both, as further described below.

First, in SCD individuals with elevated iFABP reflecting a more intense degree of intestinal injury, high levels of damage-associated molecular patterns (DAMPS) are produced that are bactericidal, leading to reduction in the intestinal microbial density that in turns decrease the LPS available for translocation across the gut barrier. Second, SCD individuals with elevated iFABP reflecting a more intense degree of intestinal injury are associated with a more rapid cell turnover of enterocytes, producing new enterocytes that are endowed with healthy tight junctions to reduce the gut permeability and LPS translocation.

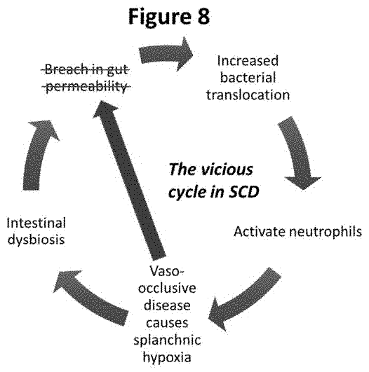

To conclude, it is found that patients with SCD have a breached intestinal barrier that allows increased translocation of intestinal bacteria/bacterial products into the systemic circulation. The correlation between the level of circulating serum LPS and activated neutrophils supports the notion that a breached intestinal barrier is responsible, at least in part, for VOC in SCD. Based on such findings, SCD can be treated by using Akkermansia muciniphila or its products to fortify the intestinal barrier to disrupt the vicious cycle of SCD, as illustrated in FIG. 8.

### Advantages

Despite the heavy burden of SCD, therapeutic options are very limited. Hydroxyurea and blood transfusion, in patients with severe acute chest syndrome and a history of stroke, remain the mainstays of therapy. Although hydroxyurea reduces acute sickle-related events, it does not appear to protect patients from cardiopulmonary complications that are the major causes of death in SCD. Hydroxyurea induces the synthesis of HbF to protect erythrocytes carrying the sickle cell hemoglobin (HbS) from sickling. The Food and Drug Agency (FDA) recently approved L-glutamine and is considering approving the P-selectin antibody, crizanlizumab, for patients with SCD. Both agents decrease the frequency of painful VOC. Whether they impact SCD-related end-organ damage and life span remain to be determined.

Gene therapy is currently in development. Two approaches are being tested. The first involves the transduction of autologous bone marrow-derived CD34 cells with the lentiviral vector containing a short-hairpin RNA targeting BCL11a to promote HbF synthesis. The second is the transfer of a modified human beta-globin gene (T87Q) into CD34+ autologous stem cells to inhibit the polymerization of HbS. The only cure currently available for SCD is allogeneic stem cell transplant (allo-SCT). However, many barriers prevent allo-SCT from being widely offered to adult SCD. This notion is exemplified by the findings of a study we carried out on 99 adult SCD patients who received care at Westchester Medical Center, New York.

Fifty five percent (55% CI: 44.75-64) of the patients were candidates for allo-SCT, based on the medical indications used by three multicenter studies (Clinicaltrials.gov Identifiers: NCT01565616, NCT02766465, and NCT03263559). We observed a correlation between the number of SCD-related complications and the hematopoietic cell transplant-comorbidity index (HCT-CI) scores (R=0.3; 95% CI: 0.04-0.52) (p=0.024). HCT-CI scores are widely applied to candidate for transplant to estimate the 2-year transplant-related mortality. Our results, therefore, suggest that SCD patients most in need of allo-SCT transplant are also those who are most at risk for dying from transplant-related complications.

In addition, 63.8% (95% CI: 50.9-75) of the patients were unemployed and 53% (CI: 40.8-65.7) relied on Medicaid for their healthcare coverage, highlighting the socio-economically disadvantaged background, often associated with lesser social support, that most of these patients came from. These factors pose barriers to allo-SCT being recommended to many of these patients. The discrepancy between the severity of disease burden and limited therapeutic options further supports the high clinical relevance and potential impact of the claimed invention in treating SCD.

For the purposes of describing and defining the present disclosure, it is noted that terms of degree (e.g., “substantially,” “slightly,” “about,” “comparable,” etc.) may be utilized herein to represent the inherent degree of uncertainty that may be attributed to any quantitative comparison, value, measurement, or other representation. Such terms of degree may also be utilized herein to represent the degree by which a quantitative representation may vary from a stated reference (e.g., about 10% or less) without resulting in a change in the basic function of the subject matter at issue. Unless otherwise stated herein, any numerical values appeared in this specification are deemed modified by a term of degree thereby reflecting their intrinsic uncertainty.

Although various embodiments of the present disclosure have been described in detail herein, one of ordinary skill in the art would readily appreciate modifications and other embodiments without departing from the spirit and scope of the present disclosure as stated in the appended claims.

What is claimed is:

1. A method for treating a patient with sickle cell disease, the method comprising:
determining a concentration of one or more biological markers in serum of the patient indicative of breach of an intestinal barrier in sickle cell disease; and
administering a micro-organismal product to the patient that restores intestinal permeability of the patient.

2. The method of claim 1, wherein administering the micro-organismal product to the patient comprises administering probiotic bacteria to the patient.

3. The method of claim 2, wherein the probiotic bacteria comprise one or more of Akkermansia muciniphila, Lachnoclostridium, Alistipes, and Pseudobutyrivibrio.

4. The method of claim 1, wherein administering the micro-organismal product to the patient comprises delivering the micro-organismal product using at least one of endoscopy, a nasogastric tube, a capsule, a drink, and a tablet.

5. The method of claim 1, wherein determining the concentration of said one or more biological markers comprises determining a concentration of one or more of intestinal fatty acid binding protein (iFABP), lipopolysaccharide (LPS), and L-selectin (CD621).

6. The method of claim 1, wherein administering the micro-organismal product to the patient comprises administering the micro-organismal product to upregulate tight-junctions of the patient's intestine.

7. A method for treating a patient with sickle cell disease, the method comprising:
determining intestinal microbial composition of the patient with sickle cell disease; and
administering a micro-organismal product to the patient, the micro-organismal product capable of restoring an intestinal microbial composition of the patient to a normal level.

8. The method of claim 7, wherein administering the micro-organismal product to the patient comprises administering probiotic bacteria to the patient.

9. The method of claim 8, wherein the probiotic bacteria comprise one or more of Akkermansia muciniphila, Lachnoclostridium, Alistipes, and Pseudobutyrivibrio.

10. The method of claim 7, wherein administering the micro-organismal product to the patient comprises delivering the micro-organismal product using at least one of endoscopy, a nasogastric tube, a capsule, a drink, and a tablet.

11. The method of claim 7, wherein determining the intestinal microbial composition comprises determining abundance of one or more of bacterial genera.

12. The method of claim 11, wherein the bacterial genera include Pseudobutyrivibrio, Faecalibacterium, Subdoligranulum, Prevotella_9, Alistipes, and Escherichia-Shigella.
